(19) United States
(12) Patent Application Publication
(10) Pub. No.: US 2026/0250440 A1
(43) Pub. Date: Aug. 27, 2026

(54) NOVEL VINYL ETHER POLYMERS AND COPOLYMERS AND PROCESSES FOR PRODUCING THE SAME

(71) Applicant: ExxonMobil Technology and Engineering Company, Spring, TX (US)

(72) Inventors: Ozcan Altintas, Franklin Park, NJ (US); Ranjan Tripathy, Sugar Land, TX (US); Phillip K. Logan, II, North Plainfield, NJ (US); Pamela J. Wright, Easton, PA (US); Adam B. Burns, Maplewood, NJ (US); Syamal Tallury, Houston, TX (US)

(21) Appl. No.: 19/546,837

(22) Filed: Feb. 23, 2026

Related U.S. Application Data
(60) Provisional application No. 63/762,911, filed on Feb. 25, 2025.

(51) Int. Cl. C08F 216/18; C07C 43/16; C08F 234/02

(52) U.S. Cl. CPC C08F 216/18; C07C 43/16; C08F 234/02; C08F 2800/20

(57) ABSTRACT

Processes of making copolymers of vinyl ether monomer and cyclic viny ether monomer are provided herein. The copolymers are produced by cationic polymerization with an initiator catalyst under ambient conditions. The vinyl ether dihydrofuran copolymers have a heat distortion temperature of at least 70° C. and a glass transition temperature of at least 65° C. providing excellent mechanical properties.

### CROSS-REFERENCE TO RELATED APPLICATIONS

This application claims priority to and the benefit of U.S. Provisional Application No. 63/762,911, entitled “Novel Vinyl Ether Polymers and Copolymers and Processes for Producing the Same,” having a filing date of Feb. 25, 2025, the disclosure of which is incorporated herein by reference in its entirety.

### FIELD

The present disclosure is related to polymers of vinyl ether or cyclic vinyl ether and copolymers of vinyl ether or cyclic vinyl ether and processes to produce the same.

### BACKGROUND

Vinyl ethers are useful as synthetic building blocks when copolymerized with other monomers. Vinyl ether monomers polymerized with 2,3-dihydrofuran (“DHF”) monomers provide copolymers having a DHF backbone and associated degradation potential, making the 2,3-dihydrofuran vinyl ether copolymers useful in different applications particularly where biodegradability is desired.

Vinyl ethers are prepared from primary and secondary alcohols while cyclic vinyl ethers can be made from diols. Due to the specialized chemical processes required to synthesize vinyl ethers and the cost and availability of raw materials, production of vinyl ethers, however, is expensive. Moreover, for many applications, high purity levels are required.

Furthermore, polymerization of different monomers is a versatile approach to synthesize polymers having improved properties. However, such processing can be a challenge for cost effective production of amorphous thermoplastics that are biodegradable and useful for products which require specific mechanical properties such as improved heat distortion temperature and without comprising the tensile/flexural strength and modulus.

### SUMMARY

Provided herein are methods of making copolymers by cationic polymerization of a vinyl ether monomer and a 2,3-dihydrofuran monomer in an initiator catalyst and a solvent under ambient conditions to produce a copolymer having a heat distortion temperature of at least 70° C. and a glass transition temperature of at least 65° C.

Further provided are methods of cationic polymerizing of vinyl ether and 2,3 dihydrofuran in a PCCP initiator catalyst under ambient conditions to produce a dihydrofuran-vinyl ether copolymer. These methods are an improvement over prior art processes by providing DIHF-VE copolymers having a heat distortion temperature of at least 70° C. and a glass transition temperature of 65° C.

In addition, provided herein are processes for making a polymer by combining an EXAAL alcohol, potassium hydroxide and water to produce a mixture. Calcium carbide is added to the mixture and the mixture is heated to 120° C. and stirred for at least 15 hours to produce an EXXAL vinyl ether that is polymerized via cationic polymerization in batch under ambient conditions without a metal ion to produce the polymer.

These and other features and attributes of the processes and polymer compositions of the present disclosure and their advantageous applications and/or uses will be apparent from the detailed description which follows.

### DESCRIPTION OF THE DRAWINGS

To assist those of ordinary skill in the relevant art in making and using the subject matter hereof, reference is made to the appended drawings, wherein:

FIG. 1A is the ¹H NMR spectrum of the EXXAL vinyl ether monomer (ExVE) produced in Example 1 in CDCl₃ at ambient temperature.

FIG. 1B is the ¹H NMR spectrum of the EXXAL vinyl ether monomer (ExVE) produced in Example 1 using the alternate preparation procedure.

FIG. 2 is the ¹H NMR spectra of the polymers PDHF (P1), PVEx (P2), and P(DHF-co-VEx) (P3) produced in Example 1 in CDCl₃ at ambient temperature.

FIG. 3 shows the size exclusion chromatography traces of PDHF (P1), P(ExVE) (P2), and P(DHF-co-ExVE) (P3) polymers of Example 1.

FIG. 4 shows the TGA curves of PDHF (P1), P(ExVE) (P2), and P(DHF-co-ExVE) (P3) polymers of Example 1 under a nitrogen atmosphere with a heating rate of 10° C. min-.

FIG. 5 are the DSC curves of PDHF (P1), P(ExVE) (P2), and P(DHF-co-ExVE) (P3) polymers of Example 1 under a nitrogen atmosphere with a heating rate of 10° C. min-.

FIG. 6 is the ¹H NMR spectrum of isobutyl vinyl ether-acetic acid THFA adduct of Example 2.

FIG. 7 shows property mapping of amorphous thermoplastics made with the copolymers produced by the present processes and acrylonitrile butadiene styrene (“ABS”).

### DETAILED DESCRIPTION

### Definitions

As used herein, Mn is number average molecular weight, Mw is weight average molecular weight, Mz is z average molecular weight, wt. % is weight percent, and mol % is mole percent. Molecular weight distribution (MWD), also referred to as polydispersity index (PDI), is the value of Mw divided by Mn. Unless otherwise noted, all molecular weight units (e.g., Mw, Mn, Mz) are presented in grams per mole (“g/mol”).

The term “isomer” or “isomerization” refers to a single or multiple rearrangements of carbon bonds without moving carbon atoms in a polymer chain. An example of such transformation includes the migration of the bonds and hydrogen atoms in a-C(1)H2-C(2)H2-C(3)H═C(4)H-unit to the —C(1)H═C(2)H—C(3)H2-C(4)H2- fragment.

An “olefin” is alternatively referred to as “alkene” and is a linear, branched, or cyclic compound of carbon and hydrogen having at least one double bond. For purposes of the present specification, when a polymer or copolymer is referred to as comprising an olefin, the olefin present in such polymer or copolymer is the polymerized form of the olefin.

As referred to herein, a “polymer” has two or more of the same or different mer units. A homopolymer is a polymer having mer units that are the same. A “copolymer” is a polymer having two or more mer units that are different from each other. A terpolymer is a polymer having three mer units that are different from each other. Accordingly, the definition of copolymer, as used herein, includes terpolymers and the like. “Different” as used to refer to mer units indicates that the mer units differ from each other by at least one atom or are different isomerically.

As used herein, a copolymer of 2,3-dihydrofuran (“DHF”) and vinyl ether (“VE”) refers to a polymer chain made up of repeating units of both DHF and VE monomers where the polymer is formed by the copolymerization of these two molecules.

As used herein, and unless otherwise specified, the term “Cn” means hydrocarbon(s) having n carbon atom(s) per molecule, wherein n is a positive integer.

The term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n. Likewise, a “Cin-Cy” group or compound refers to a group or compound comprising carbon atoms at a total number thereof in the range from m to y.

The terms “group,” “radical,” and “substituent” may be used interchangeably.

The terms “hydrocarbyl radical,” “hydrocarbyl group,” or “hydrocarbyl” may be used interchangeably and are defined to mean a group consisting of hydrogen and carbon atoms only. Example hydrocarbyls are C1-C100 radicals that may be linear, branched, or cyclic, and when cyclic, aromatic or non-aromatic. Examples of such radicals include, but are not limited to, alkyl groups such as methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like, aryl groups, such as phenyl, benzyl, naphthyl, and the like.

The terms “alkoxy” or “alkoxide” and aryloxy or aryloxide mean an alkyl or aryl group bound to an oxygen atom, such as an alkyl ether or aryl ether group/radical connected to an oxygen atom and can include those where the alkyl group is a C1 to C10 hydrocarbyl. The alkyl group may be straight chain, branched, or cyclic. The alkyl group may be saturated or unsaturated. Examples of suitable alkoxy and aryloxy radicals can include methoxy, ethoxy, n-propoxy, iso-propoxy, n-butoxy, iso-butoxy, sec-butoxy, tert-butoxy, phenoxyl, and the like.

As provided herein, a catalyst may be described as a catalyst, a catalyst precursor, a pre-catalyst compound, a catalyst compound or a transition metal compound, and these terms are used interchangeably.

Provided herein are isomeric mixed monomers prepared from readily available chemical feeds and polymerized via cationic polymerization in batch and at ambient temperature. In an embodiment, EXXAL vinyl ether (“ExVE”) monomers are synthesized from EXXAL alcohols without metal. Alternatively, ExVE monomers are synthesized from EXXAL alcohols via trans vinylation using a metal catalyst. Once produced, the ExVE monomers can be copolymerized with 2,3-dihydrofuran via the same process, which allows tuning of polymer properties. These copolymers are referred to herein as DHF-ExVE copolymer or P(DHF-co-ExVE) polymer and offer a wide range of benefits including improved packaging barrier properties, ease of recyclability, compatibility with polyolefin for recycling enhancement, lower greenhouse gas emissions compared to incumbent materials, and a less energy-intensive manufacturing process. These features collectively provide a material that is a sustainable and efficient choice in various applications.

Also provided herein is a controlled cationic polymerization process utilizing a single-component initiation under ambient conditions useful to produce both vinyl ether and copolymers. This cationic polymerization process is a batch process or alternatively is a continuous process. In an embodiment, cationic polymerization of ExVE monomers employs pentacarbomethoxycyclopentadiene (“PCCP-H”) as an initiator/catalyst. In an embodiment, the ExVE monomer is polymerized under ambient conditions or at an ambient process temperature of from about 68° F. to about 73° F. (from about 20° C. to about 23° C.). Vinyl ether monomers are prepared without a metal or metal catalyst. In an embodiment, vinyl ether monomers are prepared from EXXAL alcohols in the presence of acetylene, a low boiling point solvent and without a metal or metal catalyst. In an embodiment, the process of producing the vinyl ether monomers does not require a metal or metal catalyst.

This process has cost advantages over the prior art. The cost of the polymer produced is reduced by utilizing multicomponent vinyl ethers derived directly from readily available feedstocks. The cost advantage comes from direct functionalization of EXXAL alcohols without the need for the customary subsequent isomeric purification. This attractive approach not only provides a cost-advantage but also allows utilization of readily available feeds to create value added polymer with tunable properties.

As an alternative process, polymerization of linear and cyclic vinyl ethers is performed in a methyl chloride (“MeCl”) initiated in the presence of an initiator/Lewis acid combination in a batch or continuous process. In an embodiment, this process utilizes an initiator, 2-tetrahydrofuranyl acetate (“THFA”) in presence of proton trap (2,6-di-tert-butylpyridine) and is co-initiated with an appropriate Lewis Acid such as ethyl aluminum dichloride, EtAlCl₂ (“EADC”) or diethyl ethyl aluminum chloride Et₂AlCl (“DEAC”) or mixture of both. The resulting copolymers have mechanical properties and heat distortion temperature superior to commercial polymers such as acrylonitrile butadiene styrene copolymers-ABS.

The glass transition temperature (“Tg”) of the copolymers produced by the present processes is at least 65° C., at least 70° C., or at least 75° C. such as 75° C. to 130° C., or at least 80° C. such as 80° C. to 110° C.

The heat distortion temperature of the copolymers produced by the present processes is at least 70° C., at least 100° C. such as between 70° C. and 110° C., between 80° C. and 100° C., and between 100° C. and 110° C.

The DHF-ExVE copolymers produced by the present processes have a molecular weight of at least 80,000 grams per mole such as between 80,000 and 220,000 grams per mole. The DHF-ExVE copolymers have comparable and/or superior mechanical properties to commercially available vinyl ether polymers.

Further, the present copolymers have an Mn from about 45,000 g/mol to about 210,000 g/mol, such as from about 45,000 g/mol to about 150,000 g/mol, such as from about 45,000 g/mol to about 100,000 g/mol.

The present copolymers have an Mw from about 70,000 g/mol to about 220,000 g/mol, such as from about 75,000 g/mol to about 215,000 g/mol, such as from about 80,000 g/mol to about 210,000 g/mol, such as from about 85,000 g/mol to about 205,000 g/mol and such as 90,000 g/mol to about 200,000 g/mol.

In at least one embodiment, the present copolymers have an Mw/Mn (PDI) value from about 1.5 to about 3.5, such as about 1 to about 1.9, such as from about 1.8 to about 3.3.

General Synthesis of Producing Vinyl Ethers from Alcohols

As previously reported, the vinylation of primary and secondary alcohols can be achieved in good yields using calcium carbide CaC₂ (as a source of acetylene) under basic catalytic conditions. Matake, R. et al., Synthesis of Vinyl Ethers of Alcohols Using Calcium Carbide Under Superbasic Catalytic Condition (KOH/DMSO), Green Chem., 2016, 18, 2614-2618. In this process, vinyl ether monomers are produced from benzyl alcohols 1a using calcium carbide CaC₂ (as a source of acetylene) under catalytic conditions with KOH and water in DMSO to provide the vinyl ether product 2a as shown below in Scheme 1.

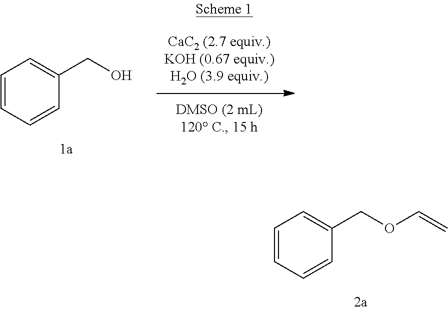

### Id,

First, a mixture of benzyl alcohol, KOH and water in DMSO is prepared. After stirring the mixture at room temperature for at least 30 minutes, calcium carbide is added and the process sealed to avoid excess pressure due to generated acetylene. The mixture is then heated to about 120° C. and stirred for at least 15 hours. Then the mixture is filtered and washed with an organic solvent such as hexane/ethyl acetate. The filtrate is evaporated under a vacuum and the resulting residue purified by column chromatography to provide a vinyl ethyl monomer product as a pale-yellow oil. Id.

Alternatively, vinylation of various alcohols can be achieved by using vinyl acetate and an iridium catalyst. Okimoto, Y. et al., Development of a Highly Efficient Catalytic Method for the Synthesis of Vinyl Ethers, J. Am. Chem. Soc., 2002, 124(8), 1590-1591. In this process, a mixture of alcohol, vinyl acetate, an iridium metal complex and an additive are mixed in toluene at elevated temperature to give vinyl ether products in high yields. In a similar reaction, Spiegelberg and coworkers demonstrate the use of bis(cyclooctadiene)iridium tetrafluoroborate catalyst, an alternative green solvent (2-methyl tetrahydrofuran) and appropriate choice of the base additive to suppress the formation of ester impurities, Spiegelberg, B. et al., Use of Iridium-Catalyzed Transfer Vinylation for the Synthesis of Bio-Based (bis)-Vinyl Ethers Adv. Synth. Catal. 2022, 364, 1251-1263.

### EXXAL™ Branched Alcohols (“EXXALs”)

Commercially available EXXAL™ branched alcohols are mixtures of long-chain, primary aliphatic branched alcohols, secondary aliphatic branched alcohols and isomers thereof. For example, EXXAL™ 11 includes C₁₀, C₁₁, and C₁₂ hydrocarbons, has about 87 wt. % of C₁₁ hydrocarbons and has an average branching number of about 2.20. Tables 1A and 1B below provide carbon number distributions and average branching of several EXXAL™ branched alcohols.

TABLE 1A

| | Average Carbon Number Distribution by GC (wt. %) | | | | | | | | | Average |
|---|---|---|---|---|---|---|---|---|---|---|
| | C₆ | C₇ | C₈ | C₉ | C₁₀ | C₁₁ | C₁₂ | C₁₃ | C₁₄ | Branching |
| EXXAL ™ 8 | <0.1 | 1.8 | 92.7 | 5.2 | 0.2 | | | | | 1.61 |
| EXXAL ™ 9 | | | 3 | 77.1 | 18.8 | 1.1 | | | | 1.87 |
| EXXAL ™ 10 | | | 0.1 | 6.4 | 88.2 | 5.2 | | | | 2.06 |
| EXXAL ™ 11 | | | | | 6.7 | 87 | 6.3 | | | 2.20 |
| EXXAL ™ 13 | | | | 0.17 | 0.3 | 1.4 | 21.5 | 70.1 | 6.7 | 3.07 |

TABLE 1B

Spec Limits Max (wt. %)

| | | | |
|---|---|---|---|
| EXXAL ™ 8 | C₆ + C₁₀⁺ | C₇ | C₉ |
| | 2.0 | 3.5 | 2.0-9.0 |
| EXXAL ™ 9 | C₈ | C₁₀ | C₁₁⁺ |
| | 6.0 | 18-22 | 2.5 |
| EXXAL ™ 10 | C₈ | C₉ | C₁₁⁺ |
| | 0.75 | 10.0 | 7.0 |
| EXXAL ™ 11 | C₁₀⁻ | C₁₁ | C₁₂⁺ |
| | 6.7 | 87.0 | 6.3 |
| EXXAL ™ 13 | C₉ + C₁₀ | C₁₄⁺ | |
| | 2.0 | 10.0 | |

In addition to data provided in Tables 1A and 1B, the percentage a branching is estimated to be between about 10% and about 15% for each of the EXXAL™ branched alcohol mixtures. The percentage of quaternary carbons is estimated to be between about 1% and about 2% for each of the EXXAL™ branched alcohols. Furthermore, EXXAL™ 13 can have an average carbon number between about 12.6 and about 12.7, an average number of branches per molecules between about 2.90 and about 3.07 and can comprise between about 60 wt. % C₁₃ and about 70.1 wt. % C₁₃. See U.S. Patent Appl. Nos. 2011/0313090 Table 1 and 2011/0184105 Table 1, incorporated herein by reference.

Objective criteria and recognized test methods show that EXXAL™ branched alcohols and ethoxylates readily biodegrade. The test methods include EPA- and EU-approved tests such as an OECD 301F manometric respirometry test that assesses “ultimate” biodegradation, or breakdown of the substance by microorganisms, resulting in the production of carbon dioxide, water, mineral salts and new biomass. The criterion to “pass” as readily biodegradable in OECD 301F test is to reach 60% degradation in 28 days (for constituent substances it is the same within a “10-day window”). EXXAL™ branched alcohols and ethoxylates meet the OECD readily biodegradable threshold for isomeric mixtures. Specifically, both EXXAL™ 11 and EXXAL™ 13 are readily biodegradable: EXXAL™ 11 demonstrated 71% degradation in 28 days and EXXAL™ 13 demonstrated 61 degradation in 28 days, both measured according to OECD 301 F.

EXXAL™ branched alcohol mixtures contain isomers having different branching structures. As to linear chains, EXXAL™ branched alcohols' purity exceeds 9900. High-purity EXXAL™ branched alcohols exhibit reactivity typical of higher primary alcohols. Having a branched structure, EXXAL™ branched alcohols are characterized by low pour points. While linear C₁₂-C₁₄ alcohols have pour points around room temperature (20° C.), branched alcohols such as EXXAL™ 13 have pour points lower than −40° C. Lower pour points have the advantage of reducing the need for heated tanks and lines for operations in colder climates, which in turn can lower energy bills and reduce handling costs.

Table 2 immediately below provides additional physical properties of EXXAL™ branched alcohols.

TABLE 2

| | EXXAL ™ | EXXAL ™ | EXXAL ™ | EXXAL ™ | EXXAL ™ |
|---|---|---|---|---|---|
| | 8 | 9 | 10 | 11 | 13 |
| Chemical Name | Isooctanol | Isononanol | Isodecanol | Isoundecanol | Isotridecanol |
| Acid Value | <0.05 | <0.05 | <0.05 | <0.10 | <0.03 |
| Mg KOH/g | | | | | |
| ASTM D1045 | | | | | |
| Boiling Range | 186-192 | 204-214 | 218-224 | 233-239 | 255-263 |
| ° C. ASTM | | | | | |
| D1078 | | | | | |
| Carbonyl | <0.20 | <0.20 | <0.20 | <0.20 | <0.20 |
| Number | | | | | |
| Mg KOH/g | | | | | |
| ISO 1843-1 | | | | | |
| ASTM E411 | | | | | |
| Color Pt/Co | 5 | 5 | 5 | 5 | 5 |
| ASTM D5386 | | | | | |
| Density 20° C. | 0.831 | 0.835 | 0.838 | 0.841 | 0.846 |
| g/cm³ | | | | | |
| ASTM D4052 | | | | | |
| Flash Pt. | >70 | >80 | >90 | >100 | >100 |
| PMCC ° C. | | | | | |
| ASTM D93 | | | | | |
| Hydroxyl | 425 | 377 | 350 | 321 | 285 |
| Number | | | | | |
| Mg KOH/g | | | | | |
| ISO 1843-5 | | | | | |
| Pour Pt. ° C. | <−40 | <−40 | <−40 | <−40 | <−40 |
| ASTM D5950 | | | | | |
| Viscosity | 12 | 17 | 21 | 27 | 48 |
| at 20° C. Mm²/s | | | | | |
| ASTM D445 | | | | | |
| Water content | <0.1 | <0.1 | <0.1 | <0.1 | <0.1 |
| wt. % ISO | | | | | |
| 12937 | | | | | |

In addition, EXXAL™ 13 has a boiling range between about 253° C. and about 265° C., a hydroxyl number of about 285 mg KOH/g, a carbonyl number between about 0.1 mg KOH/g and about 0.2 mg KOH/g, a water content between about 0.05 wt. % and about 0.1 wt. % and a viscosity at 20° C. between about 17 mm²/s and about 48 mm²/s. See U.S. Pub. No. 2011/0184105 Table 1a, incorporated herein by reference.

Vinylation of EXXALs: Vinyl Ethers Produced from EXXALs (EXXAL Vinyl Ethers)

Linear and cyclic vinyl ethers monomers can be produced from feedstocks such as EXXAL alcohols or 1,4-butanediols and create value added polymers. The ExVE monomer is used in processes to produce poly(vinyl ethers).

As described in Example 1, vinyl ethers can be produced from EXXALs using the process of Scheme 1 (shown above) as follows

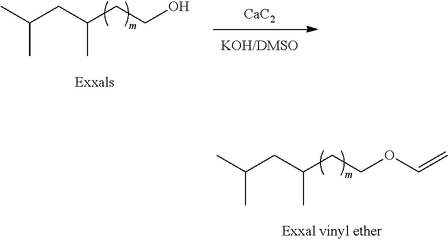

Gaseous reagents such as acetylene are generally considered as difficult reagents to handle as it involves complicated additional equipment gas regulators and detectors, high-pressure apparatus and harsh conditions. The methods described herein provide a simplified, scalable process for the synthesis of vinyl ethers i.e., without a multistep cumbersome purification.

Vinyl ethers can also be produced by transvinylation reaction with vinyl acetate as follows:

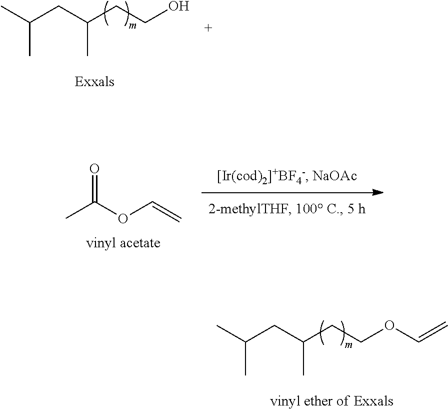

### Polymerization of Vinyl Ethers

Vinyl ethers are useful synthetic building blocks and can be copolymerized via cationic polymerization. Cationic polymerization of vinyl monomers is applicable to a relatively smaller number of monomers but is a versatile platform to produce polymers with tailored properties.

Cationic polymerization is a chain growth polymerization that involves the initiation through the formation of a carbocationic active center. A cationic initiator, typically a Lewis acid or a protonic acid compound, transfers charge to a monomer, leading to the formation of carbenium ions which are then attacked by other monomers. Polymer chain growth continues as new monomers join the chain via successive carbocation formation and propagation steps. Termination occurs through proton transfer or chain transfer to counterion, polymer, monomer, or solvent.

Controlling cationic polymerization has been a challenge due to the high reactivity of the active cationic species. However, the living polymerization technique described herein allows well-defined polymers to be synthesized with controlled molecular weight, narrow dispersity (D), and predetermined chain end functionalities.

In an embodiment, vinyl ethers are polymerized in the presence of pentacarbomethoxy cyclopentadiene (“PCCP”) and its derivatives as catalyst. In an embodiment, polymerization is performed at ambient temperature within various time intervals ranging from 1 minute to 100 hours.

Scheme 2 below shows the cationic polymerization of DHF monomer, and DHF monomer and VE monomers.

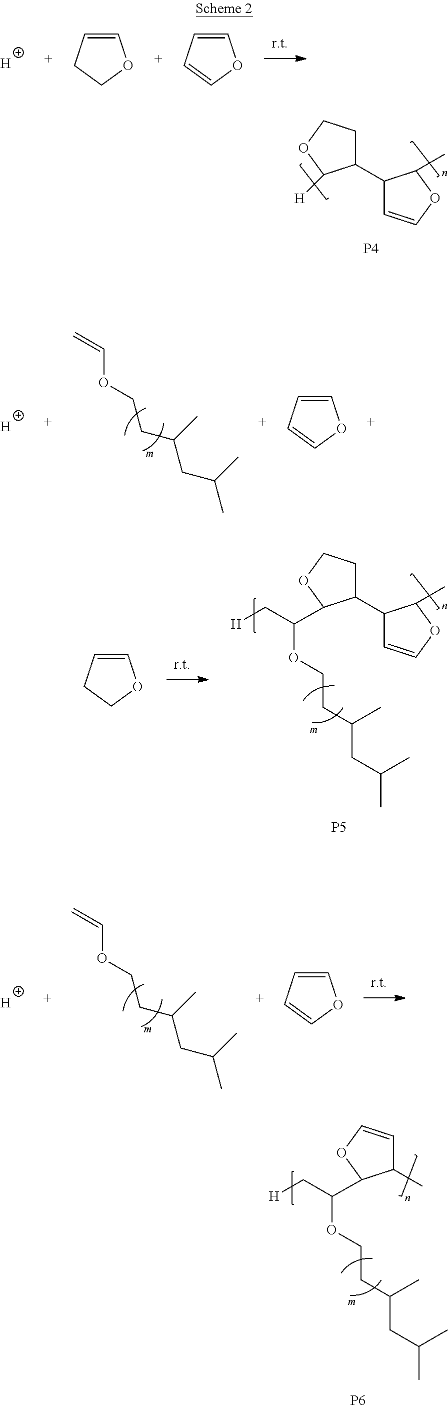

As shown below in Scheme 3, pentacarbomethoxycyclopentadiene catalysts are useful in the cationic polymerization of vinyl ethers.

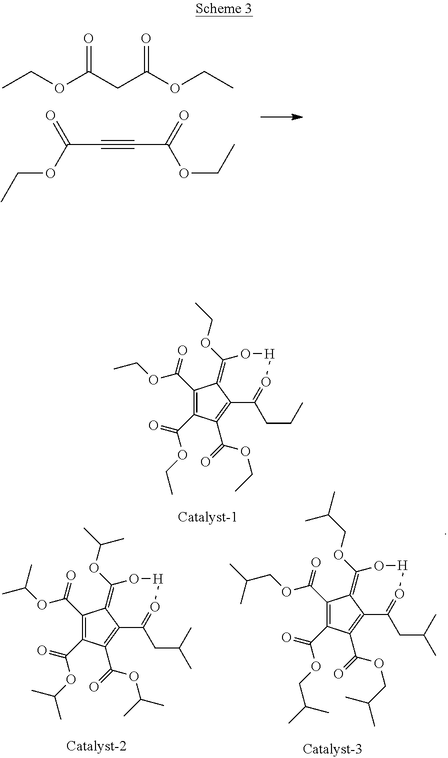

Further, methods of making the catalysts have been described. Radtke, M. et al. A Scalable, One-Pot Synthesis of 1, 2,3,4,5-Pentacarbomethoxycyclopentadiene, Synthesis (Stuttg). 51(5), 1135-1138 (2019).

Controlled cationic polymerization in a single-component initiation under ambient conditions has been described by Kottisch, V. et al., Controlled Cationic Polymerization: Single-Component Initiation Under Ambient Conditions, J Am Chem Soc., 2019, July 10; 141 (27) 10605-10609. As described, single-component acid-mediated polymerization of a variety of vinyl ethers is initiated by pentacarbomethoxycyclopentadiene (“PCCP”) and controlled by the ion pair of cyclopentadienyl anion and propagating cation. Further, as reported, this interaction allows for good chain-end fidelity and the synthesis of block copolymers. The polymerization proceeds even under ambient atmosphere and does not require rigorous purification.

Using PCCP, cyclic polymers can be polymerized via catatonic polymerization at ambient temperature as shown prophetically below in Scheme 4.

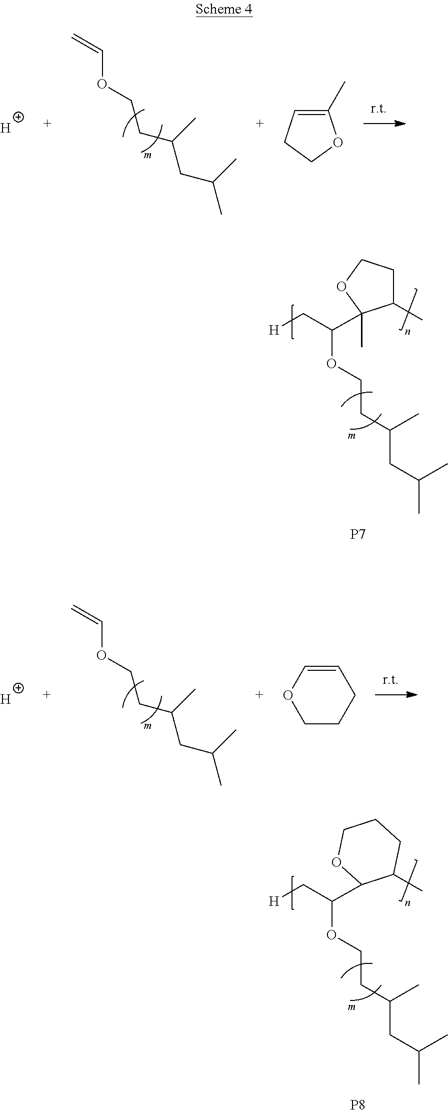

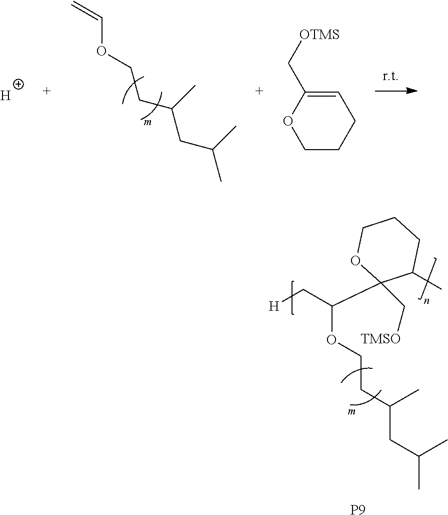

### Alternative Cationic Process to Produce DHF VE Copolymers

The present polymerizations are chain-growth polymerization and can be performed in the presence of solvent (such as dichloromethane, cyclopentyl methyl ether) or bulk. In contrast to the conventional cationic polymerization methods, which were typically carried out under inert atmospheres and low temperatures, the reaction of the present cationic polymerization techniques can go forward at room temperature, is cost-effective and environmentally friendly. As further described, cationic polymerization can be combined with other polymerization methods in a single reaction vessel, eliminating the necessity for isolation and purification during intermediate step. For example, ABS is prepared by polymerizing styrene and acrylonitrile in the presence of polybutadiene to form ABS polymers. This polymerization method, however, is different than cationic polymerization of DHF and VE. See e.g., U.S. Pat. No. 4,239,863.

Properties of Copolymers Produced with the Present Methods

Polymerization of different monomers can be a versatile approach to synthesize novel polymers with improved properties. As reported, copolymers can be synthesized through cationic polymerization for a specific arrangement of DHF and VE units depending on their relative reactivity ratios. Mondal, A. et al., Reactivity Ratio Study for the Cationic Copolymerization of Vinyl Ethers with 2,3-dihydrofuran, Journal of Macromolecular Science, Part A, 1-7 Taylor & Francis (2025) https://doi.org/10.1080/10601325.2024.2448589, accessed Jan. 15, 2025. The distribution of DHF and VE units within the polymer chain can be influenced by their respective reactivity ratios, which can be affected by factors like solvent polarity, temperature, alkyl group, and the type of Lewis acid used. Copolymers can be made with a desired composition and a wide range of material properties possible with copolymers of DHF & VE. The copolymer sequence can also depend on the substituent group (R group) in the vinyl ether, reaction temperature, solvent polarity, and the Lewis acid and its concentration.

Depending on the feed composition of DHF to VE, copolymers exhibit properties such as improved solubility, enhanced biodegradability due to the DHF units, and tailored mechanical properties. Furthermore, NMR spectroscopy is commonly used to analyze the copolymer composition and determine the distribution of DHF and VE units along the polymer chain.

As described herein, cationic polymerization is used to make polymers of various monomers. Cationic polymerization of soft segments along with rigid (hard) segments can be used to control the molecular weight and mechanical properties of the polymer/copolymer. DHF is exemplary of a rigid segment. i-propyl VE and EXXAL 9 VE are exemplary of soft segments. For amorphous thermoplastics, as described in Table 3, copolymers of DHF and VE were generated to enhance upper use temperatures and toughened the amorphous thermoplastic.

TABLE 3

| | | | | | Upper use |
|---|---|---|---|---|---|
| | Molecular | Hard/soft | | | temper- |
| Copolymer | weight | composition | Conver- | Tg | ature |
| Type | (g/mole) | (feed) | sion % | (DSC) | (TGA) |
| PDHF | 100K | 100/0 | 100 | 127 | 400 |
| PDHF-PVE | 100K | 90/10 | 95 | 110 | 400 |
| PDHF-PVE | 100K | 75/25 | 90 | 80 | 400 |
| PDHF-EXVE | 90K | 98/2 | 90 | 106 | 400 |
| PDHF-EXVE | 50K | 94/6 | 90 | 82 | 400 |

Heat is often required in manufacturing of plastics and heat distortion is critical for controlling the shape and dimensions of components during different processes such as molding. The heat distortion temperature, also known as the heat deflection temperature, is the temperature at which a polymer or plastic sample begins to deform under a specified load. Heat distortion of polymeric material impacts the functionality and appearance of materials in various applications and can cause dimensional changes in the resulting product. A higher heat distortion temperature (“HDT”) value indicates a greater resistance to heat distortion under a load. Copolymers are produced with a wide range of heat distortion temperatures which is reflective of the diverse properties.

Factors that influence heat distortion of a polymer include the arrangement of polymer chains. Crystalline polymers generally have a higher heat distortion temperature than amorphous polymers due to the organized structure. Moreover, a higher molecular weight polymer tends to have higher heat distortion because longer chains have stronger intermolecular forces. Moreover, the way a polymer is processed can affect its crystallinity and orientation and influence its heat distortion temperature. For example, heat distortion, particularly the heat deflection temperature, is crucial in molding polymers because it dictates the temperature limit at which a molded part can maintain its structural integrity and dimensional stability under a specified load. The appropriate heat distortion for a given material and specific molding application is crucial to ensure the final product can withstand the expected operating temperatures without warping or deforming.

While having a high heat distortion/heat distortion temperature is often desirable, there are applications where a lower heat distortion is acceptable or even advantageous because every product does not need to withstand high temperatures. Flexibility and ease of processing at lower temperatures are sometimes more important than extreme heat resistance. For instance, in applications like food packaging, disposable cups, or toys, lower HDT materials can be more cost-effective and easier to mold.

Selecting a polymer with an inappropriate HDT for a given application, however, can have a range of negative consequences. Utilizing a polymer with an HDT lower than the operating temperature can lead to product failure, safety risks, and increased costs due to replacements or repairs. Conversely, choosing a polymer with an unnecessarily high HDT can result in higher material and processing costs.

Likewise, a glass transition temperature (Tg) is important because it is the temperature at which a material changes from a rigid, glassy state to a more flexible, rubbery state, significantly impacting its physical properties like strength, pliability, and functionality. Depending on the application temperature, making glass transition temperature is crucial for selecting materials suitable for specific uses in various industries like plastics, electronics, and adhesives.

Depending on the application temperature, the glass transition temperature is crucial for selecting materials suitable for specific uses in various industries like plastics, electronics, and adhesives. Glass transition temperature of a polymer is particularly important in manufacturing and engineering because it influences the thermal and mechanical performance of materials. For example, in the automotive industry, components made from polymers with a well-defined Tg can withstand varying temperatures without losing their structural properties. Similarly, in aerospace applications, materials with a high Tg are preferred to ensure stability and performance at elevated temperatures. In manufacturing processes like injection molding, the Tg determines the optimal temperature range for heating and cooling the material to achieve desired shapes.

Understanding what the Tg is, and how it affects material properties, allows engineers and manufacturers to select the appropriate materials for specific applications, ensuring reliability and efficiency in their products. Thus, the glass transition temperature is a fundamental parameter in the design and application of polymer-based materials across various industries.

Cationic polymerization of soft segments along with rigid (hard) segments can be used to control the molecular weight and mechanical properties including the HDT and Tg of the polymer/copolymer. DHF is exemplary of a rigid segment. i-propyl VE and EXXAL 9 VE are exemplary of soft segments. For amorphous thermoplastics, copolymers of DIF and VE can be generated to enhance upper use temperatures and toughen the thermoplastic. In the present cationic polymerization, soft segments such as i-propyl VE or ExVE are polymerizing together with hard segments such as DHF or alkoxyl DHF.

FIG. 7 shows that poly(DHF) copolymers with VE have superior tensile, flexural and heat distortion temperature than commercial ABS.

Aspects of the disclosure are described in greater detail by way of specific examples. The following examples are offered for illustrative purposes and are not intended to limit the disclosure in any manner. Those of skill in the relevant art will readily recognize a variety of parameters can be changed or modified to yield essentially the same results.

### Additional Embodiments

Embodiment 1. A method of making a copolymer comprising the step of cationic polymerization of a vinyl ether monomer and a 2,3-dihydrofuran monomer in the presence of an initiator catalyst and a solvent under ambient conditions to produce a copolymer having a heat distortion temperature of at least 70° C. and a glass transition temperature of at least 65° C.

Embodiment 2. The process of embodiment 1, wherein the initiator catalyst is a single-component initiator.

Embodiment 3. The process of embodiment 2, where the initiator catalyst is a compound having the formula:

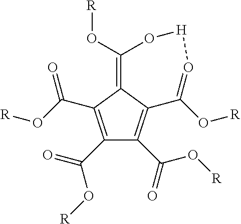

where R=C₁-C₆ alkyl.

Embodiment 4. The method of embodiment 1 wherein the copolymer is produced in the absence of a metal or metal-based Lewis acid and at a temperature of from about 20° C. to about 23° C.

Embodiment 5. The process of embodiment 1, further comprising a Lewis acid co-initiator.

Embodiment 6. The method of embodiment 1 wherein the copolymer has a molecular weight of at least 100,000 g/mole.

Embodiment 7. The method of embodiment 1, wherein the vinyl ether is an EXXAL vinyl ether having the formula:

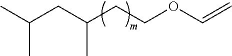

wherein m=1 to 14.

Embodiment 8. The method of embodiment 1, wherein the copolymer composition has a mass fraction of about 10 wt. % vinyl ether monomer and about 90 wt. % of dihydrofuran monomer.

Embodiment 9. The method of embodiment 1, wherein the solvent has a boiling point of at least −40° C. and less than 106° C.

Embodiment 10. The method of embodiment 9, wherein the solvent is selected from the group of dichloromethane or methyl cyclohexane or methyl chloride.

Embodiment 11. The method of embodiment 1, wherein the copolymer is thermally stable up to 360° C.

Embodiment 12. The method of embodiment 1, wherein the copolymer has a greater tensile and flexural modulus than ABS.

Embodiment 13. A method comprising cationic polymerizing vinyl ether and 2,3 dihydrofuran in the presence of a pentacarbomethoxy cyclopentadiene (PCCP) initiator catalyst under ambient conditions to produce a dihydrofuran-vinyl ether (DHF-VE) copolymer, the improvement comprising the DHF-VE copolymer having a heat distortion temperature of at least 70° C. and a glass transition temperature of 65° C.

Embodiment 14. The method of embodiment 13, wherein the glass transition temperature of the copolymer is between about 80° C. and about 110° C. and the heat distortion temperature of the copolymer is between about 70° C. and about 110° C.

Embodiment 15. The method of embodiment 13, wherein the DHF-VE copolymer is characterized by a polydispersity index (PDI) of from about 1.8 to about 3.3.

Embodiment 16. The method of embodiment 13, wherein the vinyl ether is an EXXAL viny ether (ExVE).

Embodiment 17. A process for making a polymer comprising the steps of
combining an EXAAL alcohol, potassium hydroxide and water to produce a mixture;
adding calcium carbide to the mixture;
heating the mixture to 120° C. and stirring for at least 15 hours to produce an EXXAL vinyl ether (ExVE) monomer; and
polymerizing the EXXAL vinyl ether monomer via cationic polymerization in a batch process under ambient conditions in the absence of a metal ion to produce the polymer.

Embodiment 18. The process of embodiment 17, wherein the vinyl ether monomer is polymerized with a 2,3-dihydrofuran (DHF) monomer to produce an ExVE-DHF copolymer.

Embodiment 19. A compound of the formula:

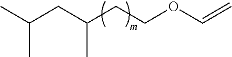

wherein m=1 to 14.

### EXAMPLES

### Example 1

### Preparation of EXXAL Vinyl Ether Monomer (“EXVE”)

Cationic polymerization of vinyl ethers was performed under ambient conditions employing pentacarbomethoxycyclopentadiene (PCCP-H) as an initiator/catalyst and using the general procedure for the synthesis of vinyl ethers from EXXALs of Scheme 1 as follows:

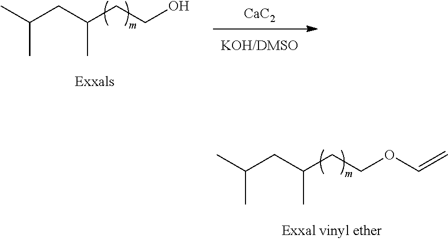

In a 250 mL two-necked round-bottom flask with a reflux condenser, a mixture of EXXAL alcohol (12.35 g), KOH (4.37 g), and water (6.46 g) in DMSO (120 mL) was prepared. After stirring at room temperature for 30 min, calcium carbide (15.75 g) was added, the flask was then sealed with a rubber septum and attached to a balloon to avoid excess pressure due to generated acetylene. The mixture was heated to 120° C. with vigorous stirring for 15 h. Then, the mixture was filtered through a pad of silica gel and washed with ethyl acetate. The filtrate was washed with brine solution. The organic phase was dried with magnesium sulfate. Ethyl acetate was evaporated under vacuum and the residue was purified by column chromatography on silica gel with hexane to give desired product as a pale-yellow oil.

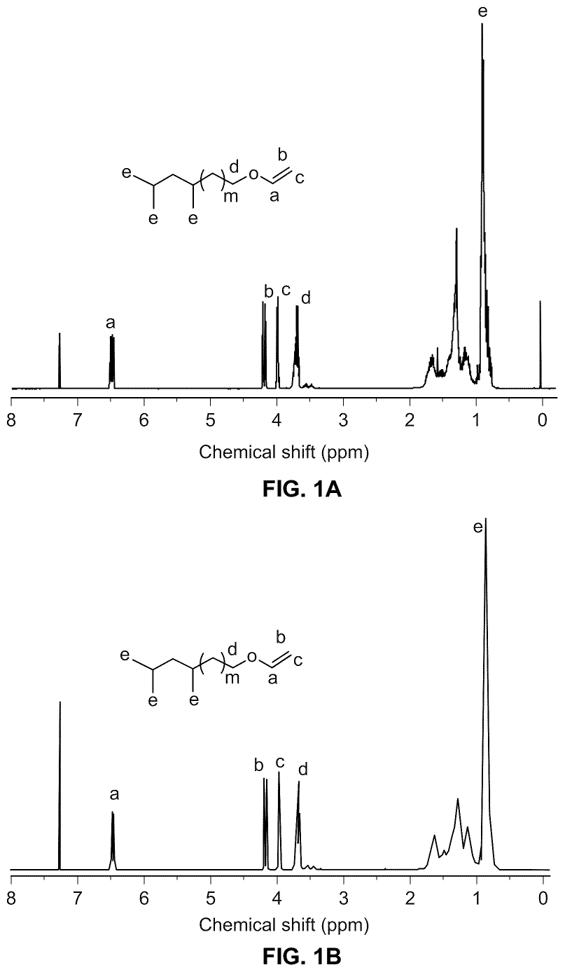

As shown in FIG. 1A, ¹H NMR spectrum of the EXXAL vinyl ether monomer has characteristic peaks at 6.46 ppm (Hₐ), 4.21 ppm (H_{b}), and 4.37 ppm (H_{c}) represent olefin protons of the monomer. The peak at 3.70 ppm (H_{d}) is related to the protons of the methylene groups adjacent to the ether unit. The peak at 6=0.91 ppm is associated with methyl protons of the monomer. ¹H NMR spectrum indicates that the EXXAL vinyl ether monomer has been successfully synthesized from EXXAL alcohols.

Alternate procedure for the Preparation of EXXAL Vinyl Ether Monomer

To a 300 mL oven dried pressure bottle equipped with a stir bar a mixture of Bis(1,5-cyclooctadiene)iridium(I) tetrafluoroborate catalyst (0.310 g), sodium acetate (1.57 g), freshly distilled 2-methyltetrahydrofuran (43 mL) and vinyl acetate (17.6 mL) were added with mixing. EXXAL10 alcohol (10.2 g) was then added to the stirring mixture. The bottle was sealed with a front seal cap and placed into a heating bath and heated to 100° C. with vigorous stirring for 18 h. The reaction flask was then removed from heat and cooled to room temperature. The solution was transferred to a round bottom flask, concentrated by rotary evaporation to remove solvent and excess vinyl acetate, then diluted a minimum of pentane (10 mL). Mixed-functionality SiliaMET gel (6.2 g) was added to the solution and this mixture was stirred overnight, after which the pentane solution was filtered. The residual gel was washed twice with 5 mL of pentane, and all pentane solutions were combined. Pentane was removed from the product by rotary evaporation. The crude product was then purified by flash chromatography with hexane. Collection and concentration of the fractions yielded the desired product as a clear colorless oil. The assignments of the ¹H NMR resonances (see FIG. 1B) are consistent with those in FIG. 1A.

### Procedure for the Polymerization of Vinyl Ethers

The polymerization was carried out under a dry nitrogen atmosphere in a glove box. The solvents and 2,3, DHF were distilled over calcium hydride. An oven-dried 50 mL vial was charged with PCCP-H and a stir bar. The vinyl ether monomers were added via syringe to the vial. An equal volume of dry solvent (dichloromethane or methyl cyclohexane) was added to solubilize the polymer through the polymerization. The reaction was stirred at ambient temperature. The polymer was purified by precipitation from methanol.

As shown in Scheme 2 above, cationic polymerization of vinyl ethers at ambient temperatures as follows:

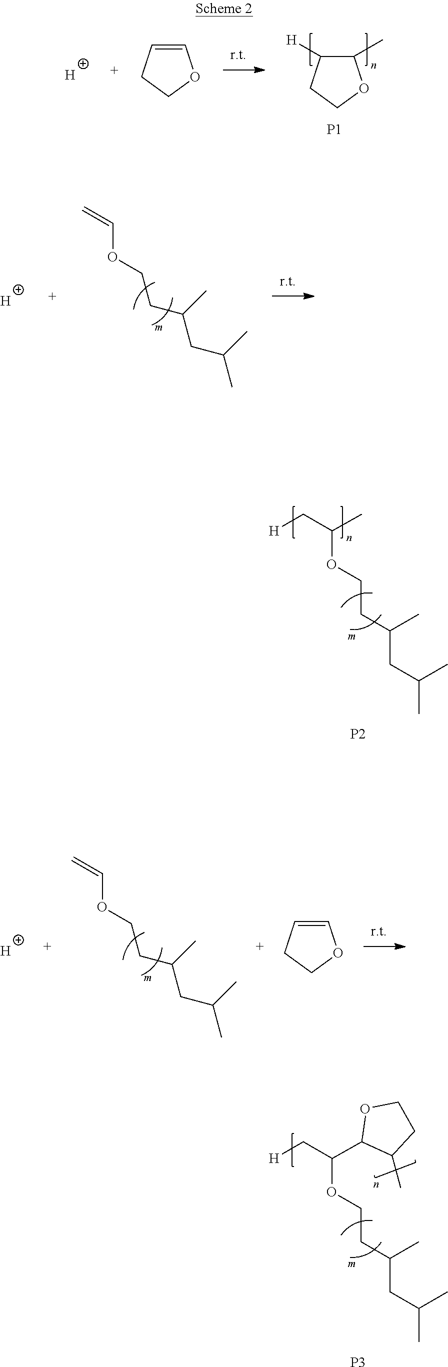

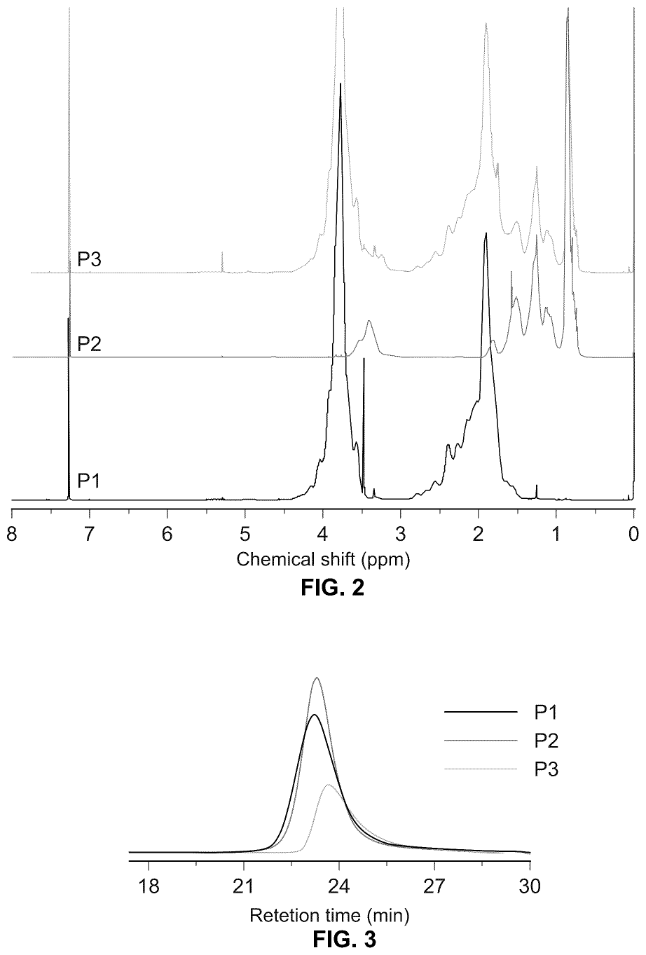

FIG. 2 shows the ¹H NMR spectra of PDHF (P1), P(ExVE) (P2), and P(DHF-co-ExVE) (P3) polymers. Characteristic chemical shifts of PDHF (P1) protons appeared at 3.78 ppm and 1.91 ppm. The resonances at 3.42 ppm assigned to the backbone protons of P(ExVE) (P2) and the resonances at 0.86 ppm assigned to the methyl protons of P(ExVE) (P2). The proton signals in the 4.45-3.12 ppm region belong to the backbone protons of P(DHF-co-ExVE) (P3) copolymers and methyl protons of the EXXAL unit appeared at 0.85 ppm suggesting incorporation of the EXXAL unit into the polymer backbone.

The molecular weights of PDHF (P1), P(ExVE) (P2), and P(DHF-co-ExVE) (P3-P7) polymers were obtained by size exclusion chromatography (SEC) using polystyrene (PS) standards and chloroform as eluent at ambient temperature. In FIG. 3, the SEC traces of the polymer demonstrated unimodal molecular weight distributions and the resulting molecular weight distributions of P1, P3 and P3 are depicted.

Table 1 provides the experimental details and molecular properties of the polymers produced including the molecular weights. As shown in Table 4, w_{1,DHF} is the feed ratio of 2,3-dihydrofuran. w_{2,ExVE} is the feed ratio of EXXAL vinyl ether and w_{3,ExVE} are the calculated mass fractions of the poly(ExVE) was determined using ¹H NMR spectroscopy.

TABLE 4

| | Catalyst | | | | | | | |
|---|---|---|---|---|---|---|---|---|
| Polymer | (Iₒ) | w_{1, DHF} | w_{2, ExVE} | w_{3, ExVE} | Mₒ/Iₒ | M_{n, RI} | M_{w, RI} | PDI |
| P1 | PCCP | 100 | 0 | — | 200 | 6400 | 20900 | 3.29 |
| P2 | PCCP | 0 | 100 | — | 200 | 4900 | 10700 | 2.19 |
| P3 | PCCP | 90 | 10 | 9.4 | 200 | 11400 | 21200 | 1.85 |
| P4 | PCCP | 90 | 10 | 9.4 | 2000 | 19300 | 44800 | 2.3 |
| P5 | PCCP | 90 | 10 | 8.7 | 5000 | 21600 | 51600 | 2.4 |
| P6 | PCCP | 90 | 10 | 9.3 | 5000 | 25700 | 63500 | 2.5 |
| P7 | PCCP/HBD | 90 | 10 | 9.3 | 5000 | 27500 | 78200 | 2.8 |

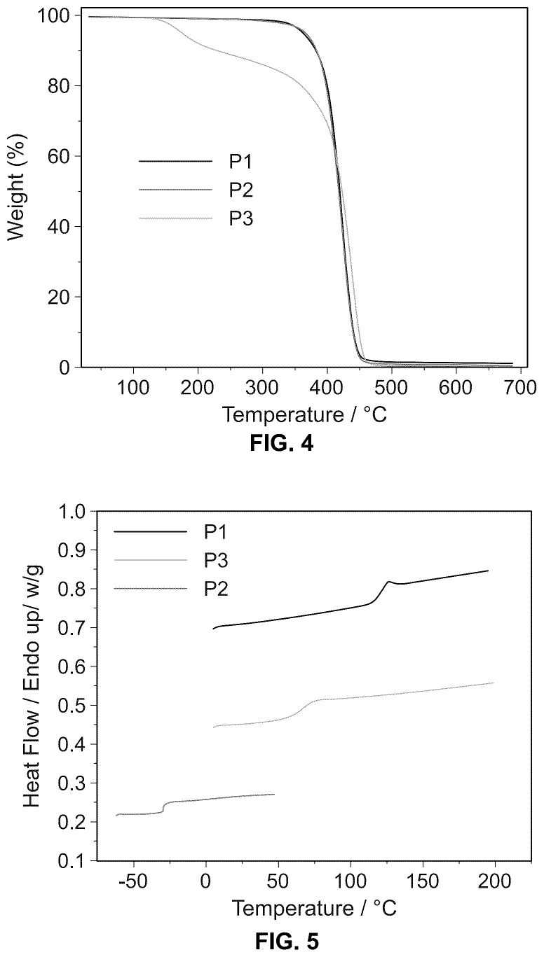

Moreover, the thermal stability of the polymers was determined by TGA at a heating rate of 10° C. min⁻¹ from room temperature to 700° C. under nitrogen atmosphere. FIG. 4 are TGA curves of PDHF (“P1”), P(ExVE) (“P2”), and P(DHF-co-ExVE) (“P3”) polymers under a nitrogen atmosphere with a heating rate of 10° C. min⁻¹. The mass loss profiles for the polymers are depicted in FIG. 4. PDHF (P1) showed less than 1% weight loss up to 340° C., then a loss of 5% weight at 360° C. P(ExVE) (P2) showed 1% weight loss at 140° C. and then 5% weight loss at 170° C. while P(DHF-co-ExVE) copolymer demonstrated 1% weight loss at 340° C. and subsequently 5% weight loss at 360° C. TGA analysis of the polymers suggest that the 2,3-dihydrofuran-based homopolymer (P1) and copolymer (P3) are thermally stable up to 360° C. The thermal stability of the poly(vinyl EXXAL) polymer was improved by copolymerization of DHF and the EXXAL vinyl monomer.

Further, the glass transition temperature (“T_{g}”) of the polymers was tested by DSC. As shown in FIG. 5, each DSC curve shows only one obvious endothermic stage, corresponding to the glass transition process. PDHF (P1) exhibited a distinct glass transition at 121° C. P(ExVE) (P2) and P(DHF-co-ExVE) (P3) exhibited glass transitions at approximately −29° C. and 65° C., respectively. The T_{g} of poly(2,3-dihydrofuran) can be tuned by introducing the EXXAL vinyl monomer.

### Example 2

In this Example, batch polymerization was carried out under a dry nitrogen atmosphere in an MBraun 150-M glove box (Innovative Technology Inc., Newburyport, Mass). Monomers of vinyl ether and 2,3 DIHF were distilled over calcium hydride and distributed to the polymerization reactors, screw top culture tubes (100 ml) at −40° C. Prior to polymerization, methyl chloride (“MeCl”) was distilled in the glovebox.

### Preparation of Lewis Acid Mixture

1 mole of ethyl aluminum dichloride, EtAlCl₂ (“EADC”) and 1 mole of diethyl ethyl aluminum chloride Et₂AlCl (“DEAC”) were mixed at room temperature inside the Glove box. The Lewis acid mixture was then placed in the bath to cool down to −40° C. prior to polymerization.

### Preparation of Initiator 2-Tetrahydrofuranyl Acetate (“THFA”)

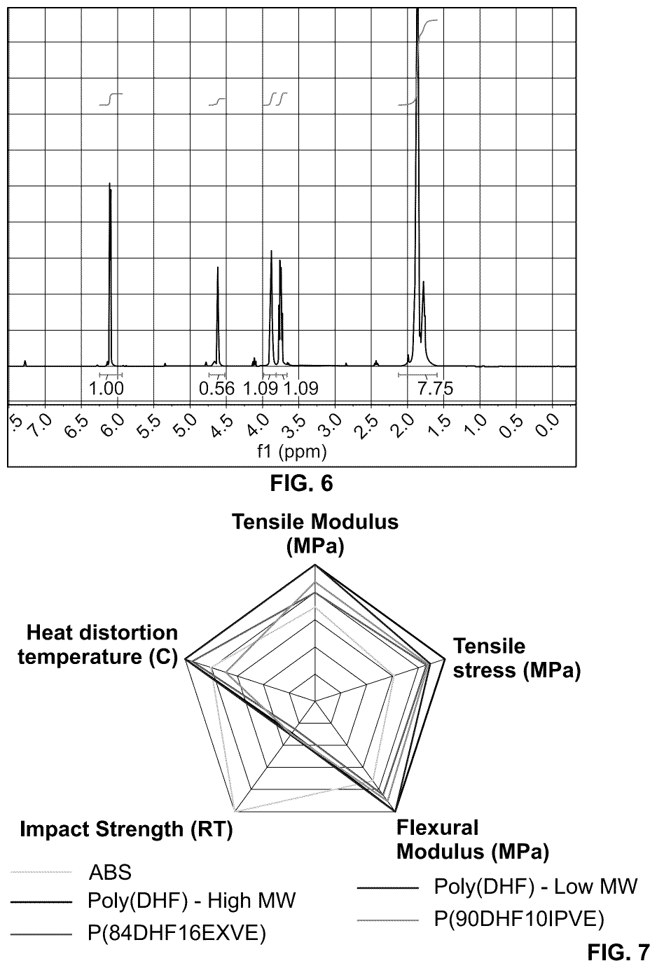

16.8 ml of 2,3 DHF was placed in a two-neck glass reactor equipped with a stirrer. 5.0 ml of acetic acid (AA, Sigma Aldrich) was added to it and the reaction was slowly heated to 60° C. The reaction mixture was maintained at 60° C. for 12 h. After the stipulated time, the reaction mixture was cooled down to room temperature. The crude product was washed six times with distilled water, till the product is neutral. Proton NMR spectroscopy was done to characterize the isobutyl vinyl ether-acetic acid THFA adduct. This THFA adduct was used as initiator for the polymerization of 2,3 DIHF & vinyl ether monomers. ¹H NMR: —CH3: (m) ppm; —CH2(m) and —CH2(m)- 1.7-2.0 ppm; —OCH2-: 3.4 and 3.5 (m) ppm; —OCHO—: 6.34 (m) ppm. FIG. 6.

Polymerizations were conducted in MeCl initiated with THFA in presence of proton trap (2,6-di-tert-butylpyridine) and co-initiated with an appropriate Lewis Acid such as ethyl aluminum dichloride, EtAlCl₂ (“EADC”) or diethyl ethyl aluminum chloride Et₂AlCl (“DEAC”) or mixture of both as described in Table 5.

TABLE 5
Polymerization Conditions

| | Monomer (M) | | | | | |
|---|---|---|---|---|---|---|
| Exp | | 2,3 | THFA | EADC | DEAC | |
| # | IPVE | DHF | (M) | (M) | (M) | DTBP |
| A | 0 | 0.95 | 0.0008 | 0.075 | 0.075 | 0.006 |
| B | 0.6 | 0.6 | 0.001 | 0.075 | 0.075 | 0.006 |
| C | 0.3 | 0.88 | 0.001 | 0.075 | 0.075 | 0.006 |
| D | 0.124 | 1.12 | 0.001 | 0.075 | 0.075 | 0.006 |
| E | 0.05 | 0.95 | 0.0007 | 0.075 | 0.075 | 0.006 |

After a predetermined time of 1 hour, polymerization was terminated by addition of 0.2 mL methanol/NH₄OH. The polymer was recovered and purified by re-precipitation from methanol solution. Conversions were determined by gravimetric analysis.

The data presented in Table 6 shows that the copolymers of DHF-IPVE can have different Tg and tensile strength depending on the feed composition or varying the hard to soft segment.

TABLE 6
Copolymer Properties

| | | | Molecular weight | | | Tensile | |
|---|---|---|---|---|---|---|---|
| | DHF/IPVE | Conversion | g/mole) (GPC) | | Tg | Strength | Elongation |
| EXP # | in feed | % | Mn | Mw | (measured) | (MPa) | at break % |
| A | 100/0 | 100 | 80,000 | 90000 | 127 | 64 | 3 |
| B | 50/50 | 100 | 94600 | 131000 | 25 | 20 | 10 |
| C | 75/25 | 95 | 146400 | 147300 | 70 | 26 | 2 |
| D | 90/10 | 95 | 99450 | 200600 | 80 | 62 | 3 |
| E | 95/5 | 100 | 99450 | 200600 | 109 | 42 | 1.5 |

### Example 3

Polymerizations were conducted in MeCl initiated with THFA in presence of proton trap (2,6-di-tert-butylpyridine) and co-initiated with an appropriate Lewis Acid, ethyl aluminum dichloride, EtAlCl₂ (“EADC”) or diethyl ethyl aluminum chloride Et₂AlCl (“DEAC”) or mixture of both with concentrations as described in Table 4. EXVE was prepared as described in Example 1.

TABLE 7
Experimental Conditions of Copolymers

| | Monomer (M) | | | | | |
|---|---|---|---|---|---|---|
| Exp | | 2,3 | THFA | EADC | DEAC | |
| # | EXVE | DHF | (M) | (M) | (M) | DTBP |
| F | 0.124 | 1.1 | 0.0005 | 0.075 | 0.075 | 0.006 |
| G | 0.18 | 0.978 | 0.0005 | 0.075 | 0.075 | 0.006 |
| H | | 2.4 | 0.00085 | 0.012 | 0.012 | 0.006 |

Table 7 shows the experimental conditions used to produce the copolymers of EXVE and DHF using THFA as initiator and the different Lewis acids. The polymerization was conducted in MeCl at −40° C. for 1 h.

The properties of the copolymers produced are provided in Table 8.

TABLE 8
Properties of Copolymers

| | | | Molecular weight | | |
|---|---|---|---|---|---|
| | DHF/EXVE | Conversion | g/mole) (GPC) | | Tg |
| EXP # | in feed | % | Mn | Mw | (measured) |
| F | 90/10 | 82 | 87000 | 159000 | 105 |
| G | 84/16 | 80 | 46702 | 87226 | 82 |
| H | 100 | 95 | 200000 | 222000 | 124 |

The data shows that the mechanical properties and heat distortion temperature of copolymers produced and are compared with commercial polymers (acrylonitrile butadiene styrene copolymers-ABS). The copolymers in this example have excellent mechanical properties and exceptional heat distortion temperature in comparison with commercial ABS.

TABLE 9
Comparison of Mechanical Properties of Copolymers

| | | | | | Heat |
|---|---|---|---|---|---|
| | Tensile | | Tensile | Flexural | Distortion |
| | Modulus | Elongation | Stress | Modulus | Temperature |
| Copolymers | (MPa) | at break | (MPa) | (MPa) | (° C.) |
| H | 3410 | 5 | 72 | 3700 | 106 |
| A | 3422 | 3 | 64 | 3780 | 106 |
| D | 2700 | 3 | 62 | 3410 | 100 |
| G | 3004 | 3.2 | 63 | 3200 | 73 |
| ABS | 2342 | 45 | 46 | 2649 | 85 |

### Procedures

### Heat Distortion Temperature (HDT)

This test method determines the temperature at which deformation occurs under three-point load. The HDT test method conforms to ASTM D648 covers the temperature at which deformation occurs when specimens are subjected to flexural stress under 3-point loading. The test method applies to molded materials of thicknesses of 3 mm (⅛ in). The temperature at which a deflection of 0.25 mm (0.010 in) occurs when specimens are subjected to a maximum stress of 0.455 MPa or 1.82 MPa via a load applied to the center of a specimen resting on its short edge (edgewise).

### Differential Scanning Calorimeter (Tg)

The Differential Scanning Calorimeter (Discovery 2500) from TA instruments measures the heat flow associated with phase transitions, such as melting, glass transitions, or decompositions. These transitions involve energy changes or heat capacity changes that can be detected by DSC with great sensitivity. Sample of 2-5 mg is placed in a DSC pan and closed with lid. The samples are then heated to 250° C. and equilibrated at high temperature, followed by cooling to −100° C. The first heating/cooling cycle was done to remove any thermal history of the copolymers. The samples are then heated back to 250° C. at a ramp rate of 10° C. per minute. The glass transition temperature is measured in the 2^{nd} heating cycle.

Molecular weights (Mw and Mn) were measured by Gel Permeation Chromatography using a Waters 150 gel permeation chromatograph equipped with a differential refractive index (DRI) detector. The numerical analyses were performed using the commercially available standard Gel Permeation Software Package.

### Mechanical Properties

Test specimens for mechanical property testing were injection-molded, unless otherwise specified. The testing temperature was standard laboratory temperature (23±2° C.) as specified in ASTM D618, unless otherwise specified. Instron load frames were used for tensile and flexure testing.

### Tensile Properties

Tensile properties were determined according to ASTM D638, including yield stress (also called tensile strength at yield) and yield strain (also called elongation at yield).

Injection-molded tensile bars were ASTM D638 Type I tested at a speed of 50.8 mm/min.

Flexure Properties: Flexure property of 1% secant modulus was determined according to ASTM D790A. Test specimen geometry is specified under “Molding Materials (Thermoplastics and Thermosets)” and the support span was 50.8 mm.

We claim:

1. A process of making a copolymer comprising the step of cationic polymerization of a feed of vinyl ether monomer and 2,3-dihydrofuran monomer in the presence of an initiator catalyst and a solvent under ambient conditions to produce a copolymer having a heat distortion temperature of at least 70° C. and a glass transition temperature of at least 65° C.

2. The process of claim 1, wherein the initiator catalyst is a single-component initiator.

3. The process of claim 2, where the initiator catalyst is a compound having the formula: 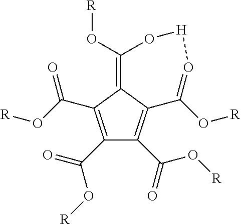
where R=C₁-C₆ alkyl.

4. The process of claim 1 wherein the copolymer is produced in the absence of a metal or metal-based Lewis acid and at a temperature of from about 20° C. to about 23° C.

5. The process of claim 1, further comprising a Lewis acid co-initiator.

6. The process of claim 1 wherein the copolymer has a molecular weight of at least 100,000 g/mole.

7. The process of claim 1, wherein the vinyl ether is an EXXAL vinyl ether having the formula: 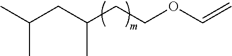
wherein m=1 to 14.

8. The process of claim 1, wherein the copolymer has a mass fraction of from 2 wt. % to 50 wt. % vinyl ether monomer.

9. The process of claim 1, wherein the solvent has a boiling point of at least −40° C. and less than 106° C.

10. The process of claim 9, wherein the solvent is selected from the group of dichloromethane or methyl cyclohexane or methyl chloride.

11. The process of claim 1, wherein the copolymer is thermally stable up to 360° C.

12. The process of claim 1, wherein the copolymer has a greater tensile stress greater than 50 MPa and flexural modulus greater than 3000 MPa.

13. A process comprising cationic polymerizing vinyl ether and 2,3 dihydrofuran in the presence of a pentacarbomethoxy cyclopentadiene (PCCP) initiator catalyst under ambient conditions to produce a dihydrofuran-vinyl ether (DHF-VE) copolymer, the improvement comprising the DHF-VE copolymer having a heat distortion temperature of at least 70° C. and a glass transition temperature of 65° C.

14. The process of claim 13, wherein the glass transition temperature of the copolymer is between about 80° C. and about 110° C. and the heat distortion temperature of the copolymer is between about 70° C. and about 110° C.

15. The process of claim 13, wherein the DHF-VE copolymer is characterized by a polydispersity index (PDI) of from about 1.8 to about 3.3.

16. The process of claim 13, wherein the vinyl ether is an EXXAL vinyl ether (ExVE).

17. A process for making a polymer comprising the steps of
combining an EXAAL alcohol, potassium hydroxide and water to produce a mixture;
adding calcium carbide to the mixture;
heating the mixture to 120° C. and stirring for at least 15 hours to produce an EXXAL vinyl ether (ExVE) monomer; and
polymerizing the EXXAL vinyl ether monomer via cationic polymerization in a batch process under ambient conditions in the absence of a metal ion to produce the polymer.

18. The process of claim 17, wherein the vinyl ether monomer is polymerized with a 2,3-dihydrofuran (DHF) monomer to produce an ExVE-DHF copolymer.

19. A compound of the formula: 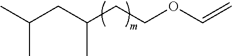
wherein m=1 to 14.
